(19) United States
(12) Patent Application Publication
(10) Pub. No.: US 2025/0064712 A1
(43) Pub. Date: Feb. 27, 2025

(54) METHOD FOR AMPLIFYING EXPRESSION OF EPIDERMAL BARRIER FUNCTION-RELATED GENE

(71) Applicant: J-Network, Inc., Newport Beach, CA (US)

(72) Inventors: Tatsuro MIYOSHI, Newport Beach, CA (US); Brian Charles KELLER, Newport Beach, CA (US); Akira KODAMA, Newport Beach, CA (US)

(21) Appl. No.: 18/744,384

(22) Filed: Jun. 14, 2024

(30) Foreign Application Priority Data
Aug. 21, 2023 (JP) 2023-133848

(51) Int. Cl. A61K 8/86; A61K 8/67; A61Q 19/08

(52) U.S. Cl. CPC A61K 8/86; A61K 8/671; A61Q 19/08

(57) ABSTRACT

An action of amplifying expression of an epidermal barrier function-related gene by a diacylglycerol polyethylene glycol adduct is utilized. A method for amplifying expression of an epidermal barrier function-related gene is provided. The method comprises applying a diacylglycerol PEG adduct, wherein the diacylglycerol PEG adduct is at least one of a group consisting of PEG-12 glycerol dimyristate (GDM12), PEG-12 glycerol distearate (GDS12), PEG-23 glycerol distearate (GDS23), PEG-23 glycerol dipalmitate (GDP23), and PEG-12 glycerol dioleate (GDO12).

### FIELD

The preset invention relates to a method for amplifying expression of an epidermal barrier function-related gene.

### BACKGROUND

Japanese Patent No. 4497765 (Self-forming, thermodynamically stable liposomes and their applications) discloses a preparation method in which a diacylglycerol polyethylene glycol adduct (hereinafter referred to as “diacylglycerol PEG adduct”) is utilized as a lipid molecule and mixed with water or a surfactant to spontaneously form a vesicle. Such a vesicle is used in a drug delivery system in which a target substance such as a protein or a drug is encapsulated in or bound to the inside or surface of the vesicle and delivered to cells in a living body. A vesicle composed of a diacylglycerol PEG adduct has a form in which the surface thereof is covered with a hydrophilic PEG chain, and has good permeability into a living body and good stability in blood.

Japanese Patent No. 6297737 (Preparing method for positively-electrified charged noisome, and charged niosome) describes that a charged element is bound to the surface of a vesicle composed of a diacylglycerol PEG adduct and positively-electrified, thereby improving permeability and retention of the vesicle in the stratum corneum of the epidermis.

The vesicle in a drug delivery system has been recognized simply as a carrier of a drug which is a target substance. In recent years, it has been known that the molecule itself of the diacylglycerol PEG adduct derived from the vesicle degraded in the living body also exerts a useful action in the living body.

Japanese Patent No. 6805385 (Expression enhancer of moisturizing related substances in epidermis) discloses that a diacylglycerol PEG adduct contributes to enhancement of expression of moisturizing related substances such as profilaggrin, filaggrin, and natural moisturizing factor NMF in the epidermis. In addition, Japanese Patent No. 6860739 (Expression enhancer of antioxidant in epidermis) discloses that a diacylglycerol PEG adduct contributes to enhancement of expression of antioxidant-related substances such as Nrf2 and PPARG, which are oxidative stress-response genes, as well as NQO-1, CAT, and HMOX1, which are antioxidant enzymes, in the epidermis.

Meanwhile, in the epidermis, various proteins or enzymes that constitute the stratum corneum of the epidermis or are involved in epidermal cell proliferation are responsible for the epidermal barrier function, which functions to prevent the invasion of bacteria, inflammatory substances, allergens, and the like from the outside into the epidermis and to maintain skin homeostasis by maintaining moisture of the skin to prevent drying of the skin.

For example, PPARα, one of the nuclear receptors, is a protein that controls gene expression related to the regulation of lipid metabolism and inflammatory responses in the skin (Japanese Patent Application Laid-open No. 2018-48103 (Skin quality improving agent)). An extremely large number of synthetic or natural substances are known as ligands that activate PPARα.

Further, for example, RARγ, which is a nuclear receptor of retinoic acid (vitamin A), is a protein that controls gene expression related to normal differentiation and regeneration of epidermal cells (Japanese Patent Application National Publication No. 2014-528472 (Anti-aging composition through activation of retinoic acid receptor)).

For example, loricrin and involucrin are precursor proteins of the cornified envelope enclosing the corneocytes, are produced according to the differentiation of the epidermal cells, promote the cornification of the epidermis, and stabilize the stratum corneum (Japanese Patent Application Laid-open No. 2020-160028 (Screening method of skin barrier function improvement agent)).

In addition, for example, hyaluronan synthase 3 maintains homeostasis of the epidermis by synthesizing hyaluronic acid in the epidermis and controlling moisture (Japanese Patent Application Laid-open No. 2011-98983 (Hyaluronic acid production enhancer)).

In addition, for example, ceramide synthases 2 and 3 produce ceramide, which is an intercellular lipid playing an important role in maintaining the epidermal barrier function (Japanese Patent Application Laid-open No. 2016-23155 (Ceramide production promoter and skin external preparation)).

Also, for example, cholesterol sulfotransferase controls the metabolism of cholesterol sulfate in the skin and enhances the epidermal barrier function and moisturizing (Japanese Patent Application National Publication No. 2019-529515).

As described above, the diacylglycerol PEG adduct has the action of the enhancement of expression of a moisturizing related substance described in Japanese Patent No. 6805385 and the action of the enhancement of expression of an antioxidant described in Japanese Patent No. 6860739. These actions contribute to the increase of moisturizing factors in the epidermis and the alleviation of oxidative stress generated in the epidermis.

However, even if the expression of moisturizing related substances or antioxidants is enhanced, when the epidermal barrier function is reduced, the increased moisturizing factors cannot be maintained, and the occurrence of oxidative stress cannot be prevented. How diacylglycerol PEG adducts are related to the epidermal barrier function is little known.

An object of the present invention is to utilize a newly found property associated with an epidermal barrier function in diacylglycerol PEG adducts, and in particular to utilize a property that amplifies the expression of epidermal barrier function-related genes.

### SUMMARY

In order to achieve the above object, the present invention provides the following configurations.

An aspect of the present invention provides a method for amplifying expression of an epidermal barrier function-related gene, the method comprising applying a diacylglycerol PEG adduct having a following structural formula;
in which number of carbon atoms of R in a long-chain fatty acid is in a range of 11 to 23 and n in a polyethylene glycol chain is in a range of 11 to 46,
wherein the diacylglycerol PEG adduct is at least one of a group consisting of PEG-12 glycerol dimyristate (GDM12), PEG-12 glycerol distearate (GDS12), PEG-23 glycerol distearate (GDS23), PEG-23 glycerol dipalmitate (GDP23), and PEG-12 glycerol dioleate (GDO12).

Another aspect of the present invention provides a method for using an active ingredient for amplifying expression of an epidermal barrier function-related gene in a cosmetic or a skin external preparation, the method comprising using, as the active ingredient, a diacylglycerol PEG adduct having a following structural formula;
in which number of carbon atoms of R in a long-chain fatty acid is in a range of 11 to 23 and n in a polyethylene glycol chain is in a range of 11 to 46,
wherein the diacylglycerol PEG adduct is at least one of a group consisting of PEG-12 glycerol dimyristate (GDM12), PEG-12 glycerol distearate (GDS12), PEG-23 glycerol distearate (GDS23), PEG-23 glycerol dipalmitate (GDP23), and PEG-12 glycerol dioleate (GD012).

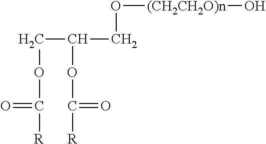

It is preferred that the diacylglycerol PEG adduct permeates into epidermis in a solution state or a vesicle state.

It is preferred that the cosmetic or the skin external preparation further comprises at least one of a group consisting of retinoic acid, retinol, a retinol derivative, and a retinoic acid derivative.

According to the present invention, amplification of expression of epidermal barrier function-related genes is realized by diacylglycerol PEG adducts.

Further, according to the present invention, amplification of expression of epidermal barrier function-related genes is realized by a cosmetic or a skin external preparation containing a diacylglycerol PEG adduct as an active ingredient.

### BRIEF DESCRIPTION OF DRAWINGS

FIG. 1 is a graph illustrating relative expression levels of PPARA genes in GDS23;

FIG. 2 is a graph illustrating relative expression levels of PPARA genes in GDM12;

FIG. 3 is a graph illustrating relative expression levels of RARG genes in GDS23;

FIG. 4 is a graph illustrating relative expression levels of RARG genes in GDM12;

FIG. 5 is a graph illustrating relative expression levels of LOR genes in GDS23;

FIG. 6 is a graph illustrating relative expression levels of LOR genes in GDM12;

FIG. 7 is a graph illustrating relative expression levels of IVL genes in GDM12;

FIG. 8 is a graph illustrating relative expression levels of HAS3 genes in GDS23;

FIG. 9 is a graph illustrating relative expression levels of HAS3 genes in GDM12;

FIG. 10 is a graph illustrating relative expression levels of CERS2 genes in GDS23;

FIG. 11 is a graph illustrating relative expression levels of CERS2 genes in GDM12;

FIG. 12 is a graph illustrating relative expression levels of CERS3 genes in GDS23;

FIG. 13 is a graph illustrating relative expression levels of SULT2B1 genes in GDS23;

FIG. 14 is a graph illustrating relative expression levels of SULT2B1 genes in GDM12; and

FIG. 15 is an image illustrating test results of the loricrin (LOR) immunostaining method (upper row) and the hematoxylin-eosin (HE) staining method (lower row) with respect to GDS23.

### DESCRIPTION OF EMBODIMENTS

Embodiments of the present invention will be described below with reference to the drawings.

The present invention has been made by utilizing a newly found property in a diacylglycerol polyethylene glycol adduct (diacylglycerol PEG adduct). The newly found property is the action of amplifying the expression of human epidermal barrier function-related genes.

The structural formula of the diacylglycerol PEG adduct which is a lipid molecule according to the present invention is schematically illustrated.

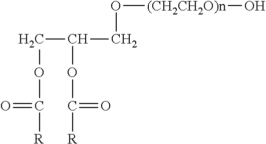

Diacylglycerol PEG adducts are composed of a glycerol skeleton (CH₂CHCH₂) having three carbons, a PEG chain which is a linear polyethylene glycol bonded to one carbon at the terminal of the three carbons of the skeleton, and the same type of long chain fatty acid (COOR) bonded to each of the other two of the three carbons. Part of the PEG-chain is hydrophilic and part of the long chain fatty acid is hydrophobic.

When a specific diacylglycerol PEG adduct is described in the following description, it is referred to as “[PEG-n]+[glycerol]+[di]+[long chain fatty acid name]” based on the type of the long chain fatty acid and the number n of the PEG chain. For example, when the long chain fatty acid is myristic acid and n of the PEG chain is 12, it is “PEG-12 glycerol dimyristate”. In addition, a specific diacylglycerol PEG adduct may be further abbreviated.

The number of carbon atoms of R in the long-chain fatty acid can be in a range of 11 to 23. The long-chain fatty acids included in this range are, for example, myristic acid, palmitic acid, stearic acid, oleic acid or the like. The number n of PEG chains can be in the range of 11 to 46. Examples of the diacylglycerol PEG adducts related to the present invention include the following substances. Melting points and abbreviations are indicated in parentheses.
PEG-12 glycerol dimyristate (25.0° C.: GDM12)
PEG-12 glycerol distearate (40.0° C.: GDS12)
PEG-23 glycerol distearate (39.8° C.: GDS23)
PEG-23 glycerol dipalmitate (31.2° C.: GDP23)
PEG-12 glycerol dioleate (25.0° C.: GDO12)

The human epidermal barrier function is responsible for preventing the invasion of bacteria, inflammatory substances, allergens, and the like from the outside and avoiding various skin problems. PPARA, RARG, LOR, IVL, HAS3, CERS2, CERS3, and SULT2B1 are epidermal barrier function-related genes, the expression of which has been confirmed to be amplified by diacylglycerol PEG adducts in the present invention. These are collectively referred to herein as “epidermal barrier function-related genes”. Each of these genes encodes a corresponding protein or enzyme. Substances that activate these genes are expected in the control of skin functions.

The PPARα protein encoded by the PPARA gene is one of the homologs of the peroxisome proliferator-activated receptor PPAR, which is a nuclear receptor. PPARα has a lipolytic function, and in particular, PPARα present in the epidermis induces differentiation of keratinocytes and promotes skin lipogenesis, thereby maintaining the epidermal barrier function and inhibiting skin inflammation.

The RARγ protein encoded by the RARG gene is one of the homologues of the nuclear receptor RAR, and uses a fat-soluble vitamin, especially retinoic acid, as a ligand. RARγ has a function of maintaining homeostasis, and especially RARγ present in the epidermis has an action of controlling differentiation and regeneration of the epidermal cells.

Retinoic acid can also be exogenously administered to the skin, but it is insufficient in stability to light and heat and skin permeability. Accordingly, retinol, retinol derivatives, or retinoic acid derivatives improved in these properties may be administered to the skin as an ingredient of a cosmetic or the like, for example, so that they are converted into retinoic acid in the body and function as ligands.

Retinol derivatives are compounds formed by bonding retinol with ester bonds or the like, and are hydrolyzed to produce retinol. Retinol is oxidized to retinoic acid by enzymatic action. Retinol derivatives include, for example, retinol palmitate.

Retinoic acid derivatives are compounds formed by bonding retinoic acid with ester bonds or the like, and are hydrolyzed to retinoic acid. Examples of retinoic acid derivatives include, for example, tocopheryl retinoate and hydroxypinacolone retinoate.

The loricrin and involucrin proteins encoded by the LOR and IVL genes, respectively, are expressed in between the stratum spinosum and the stratum granulosum by the differentiation of epidermal cells, and are cross-linked by enzymes with the differentiation of epidermal cells to form a cornified envelope (CE) covering corneocytes, thereby promoting keratinization of the epidermis and strengthening the barrier function of the skin.

Hyaluronan synthase encoded by the HAS3 genes synthesizes epidermal hyaluronic acid to maintain and control the amount of moisture in the skin, thereby maintaining epidermal homeostasis.

Ceramide synthase 2 and ceramide synthase 3, which are encoded by the genes of CERS2 and CERS3, respectively, synthesize ceramide, which is an intercellular lipid. Ceramide is produced in the stratum granulosum and finally forms a lamellar structure between the cells of the stratum corneum, which is responsible for the epidermal barrier function.

Cholesterol sulfotransferase, encoded by the SULT2B1 genes, controls the metabolism of cholesterol sulfate in the skin to enhance skin barrier function and moisture retention through differentiation into corneocytes.

The present inventors have found that the expression of epidermal barrier function-related genes is amplified by applying a diacylglycerol PEG adduct to human epidermis. By amplifying the expression of epidermal barrier function-related genes, the production of proteins and enzymes encoded by these genes is enhanced. This is a novel action of the diacylglycerol PEG adduct relating to the human epidermis, particularly to the epidermal barrier function, and is a novel property of the diacylglycerol PEG adduct. This property can provide effects on the epidermis, such as preventing the invasion of toxic substances, allergens, and the like from the outside, preventing the occurrence of oxidative stress in the skin due to external factors, and maintaining moisture retention ability on the skin. By exerting these effects, drying of the skin can be prevented, skin homeostasis can be maintained, and inflammatory responses can be suppressed. This also leads to improvement of symptoms of chronic skin diseases such as atopic dermatitis and infection. This epidermal barrier function is not simply a physical protective effect on the epidermal surface, but an effect obtained in epidermal cells.

The present invention provides a method for amplifying the expression of epidermal barrier function-related genes by applying a diacylglycerol PEG adduct utilizing the newly found property of the diacylglycerol PEG adduct.

The present invention also provides a method for using the diacylglycerol PEG adduct in a cosmetic or a skin external preparation as an active ingredient for amplifying the expression of epidermal barrier function-related genes.

In the present invention, when the diacylglycerol PEG adduct is applied to the human epidermis, only one kind of the diacylglycerol PEG adduct may be used, or a plurality of kinds thereof may be used in combination.

According to the present invention, the diacylglycerol PEG adduct that has reached the inside of the epidermis can amplify the expression of epidermal barrier function-related genes and increase the production amount of proteins and enzymes corresponding thereto, as compared with the case where the diacylglycerol PEG adduct is not present. As a result, the state of not only the inside of the epidermis but also the surface of the epidermis is improved. By carrying out the method of the present invention, it is possible to provide a cosmetic or a skin external preparation containing, as an active ingredient, a diacylglycerol PEG adduct as an expression amplifier for epidermal barrier function-related genes. Both the cosmetic and the skin external preparation are applied to the epidermal surface. Here, those other than the cosmetic are generally referred to as skin external preparations, and examples thereof include ointments or the like. Such a cosmetic or a skin external preparation can be provided in various forms such an aqueous solution, a milky lotion, a gel, a cream, and the like. These cosmetics or skin external preparations can contain, in addition to the diacylglycerol PEG adduct, other active ingredients and/or various ingredients that are generally contained.

As one method for allowing the diacylglycerol PEG adduct to reach the human epidermis, the diacylglycerol PEG adduct can be allowed to reach the epidermis in a state of a solution prepared by dissolving the diacylglycerol PEG adduct in water or a predetermined solvent. For example, a solution of the diacylglycerol PEG adduct having a predetermined concentration is prepared by using a phosphate-buffered saline solution PBS (-) as a solvent, and the solution is applied to the surface of the epidermis, whereby the applied solution can be allowed to penetrate into the epidermis. The applied solution permeates, for example, into the uppermost stratum corneum, further into the stratum granulosum below the stratum corneum, and further into the underlying layer. And the diacylglycerol PEG adduct amplifies the expression of epidermal barrier function-related genes in each layer within the epidermis into which the solution has penetrated.

As another method for allowing the diacylglycerol PEG adduct to reach the epidermis, the diacylglycerol PEG adduct can be allowed to reach the epidermis in a vesicle state. Such a vesicle is formed as a closed spherical shell consisting of a bilayer or multiple layers of the bilayers of the diacylglycerol PEG adduct, and hydrophilic PEG chains are arranged on the surface of the outermost layer. A vesicle of the diacylglycerol PEG adduct can be prepared and applied to the epidermal surface to penetrate into the epidermis. After the vesicle of the diacylglycerol PEG adduct reaches the epidermis, the vesicle is decomposed and separated into individual molecules, whereby the diacylglycerol PEG adduct itself can exert its action.

In the conventional drug delivery system, the diacylglycerol PEG adduct, which is a vesicle material, has been considered as a mere carrier of the target substance. However, in the present invention, the diacylglycerol PEG adduct itself is utilized as an active ingredient. Therefore, in the present invention, the target substance incorporated into the vesicle in the ordinary drug delivery system is basically unnecessary. In the present invention, the diacylglycerol PEG adduct itself can function as an expression amplifier of epidermal barrier function-related genes by allowing the vesicle formed by mixing only water and the diacylglycerol PEG adduct to penetrate into the epidermis.

Some diacylglycerol PEG adducts spontaneously form vesicles when mixed with water at a predetermined temperature (see Japanese Patent No. 4497765 and No. 6297737). For example, by mixing and stirring 2% by mass of GDM12 or GDO12 with 98% by mass of deionized water at room temperature, a suspension of vesicles of GDM12 or GDO12 is obtained. As another example, a suspension of vesicles of GDS12 or GDS23 can be obtained by dissolving 2% by mass of GDS12 or GDS23 at 45 to 55° C., and then mixing and stirring with 98% by mass of deionized water at 45 to 55° C. As yet another example, a suspension of vesicles of GDP23 can be obtained by dissolving 2% by mass of GDP23 at 37° C. and then mixing and stirring with 98% by mass of deionized water at 37° C. The vesicles remain stable even when the suspension obtained at a temperature higher than room temperature is cooled to room temperature.

As another example, the use of a vesicle formed by mixing an aqueous solution of various substances in place of the water described above with a diacylglycerol PEG adduct and stirring the mixed solution is also included in the scope of the present invention. In this case, the substance contained in the aqueous solution may have another function.

As still another example, a case where the surface of a vesicle formed by mixing and stirring water or an aqueous solution and a diacylglycerol PEG adduct is modified with a charged element such as a cationic surfactant is also included in the scope of the present invention. Japanese Patent No. 6297737 describes that a positively charged vesicle is particularly excellent in permeability into the epidermis and retention.

Hereinafter, the relation between the application of diacylglycerol PEG adducts to epidermal cells and epidermal barrier function-related genes will be shown by test data.

### (1) Test for Amplification of Expression of Epidermal Barrier Function-Related Genes

A test for confirming the amplification of mRNA expression of each epidermal barrier function-related gene was performed.

### (1-1) Test Method

Normal human epidermal keratinocytes (NHEKs) were seeded on a 96-well plate at a cell density of 2.0×10⁴cells/well using HuMedia-KG2 medium (manufactured by Kurabo Industries Ltd.), and cultured under the conditions of 37° C. and 5% CO₂ for 24 hours.

Thereafter, a diacylglycerol PEG adduct was added thereto using HuMedia-KB2 medium (manufactured by Kurabo Industries Ltd.), and each was cultured under the conditions of 37° C. and 5% CO₂ for a predetermined time. Table 1 represents the target gene, the types of diacylglycerol PEG adduct added, the adding amount, and the culture time. A control (no addition) was also cultured under the same conditions.

TABLE 1

| Target gene | Types | Amount(μM) | Culture time (hr) |
|---|---|---|---|
| PPARA | GDS23 | 50 | 24, 30, 36 |
| | GDM12 | 100 | 24, 30 |
| RARG | GDS23 | 50 | 24, 30, 36 |
| | GDM12 | 100 | 24, 30 |
| LOR | GDS23 | 50 | 24, 30, 36 |
| | GDM12 | 100 | 30 |
| IVL | GDM12 | 100 | 30 |
| HAS3 | GDS23 | 50 | 24, 30, 48 |
| | GDM12 | 100 | 24, 30 |
| CER2 | GDS23 | 50 | 24, 30, 36 |
| | GDM12 | 100 | 24, 30 |
| CER3 | GDS23 | 50 | 36 |
| SULT2B1 | GDS23 | 50 | 24 |
| | GDM12 | 100 | 30 |

After the cells were cultured for each predetermined time, RNA was extracted from the cells of each sample and control. The extracted RNA was reverse-transcribed to prepare cDNA, and the mRNA of the target gene was quantified by quantitative real-time PCR expression analysis. GAPDH (glyceraldehyde 3-phosphate dehydrogenase) was used as an internal standard.

In the analysis, the mRNA expression level of each target gene was corrected with the value of the expression level of GAPDH, which is an internal standard in the same sample, and the correction value of the sample was calculated as a relative expression level when the correction value of the control was set to 1.

### (1-2) Test Results

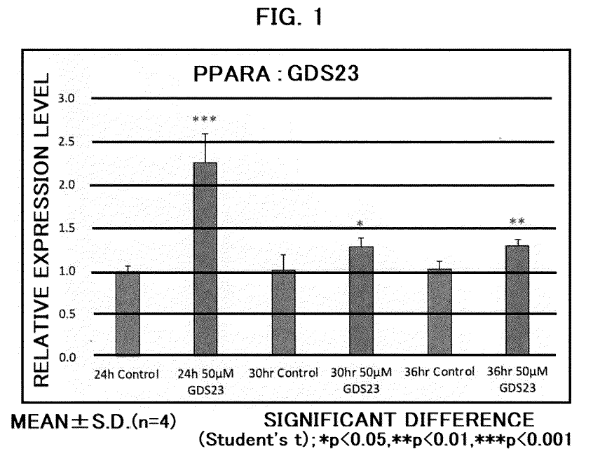

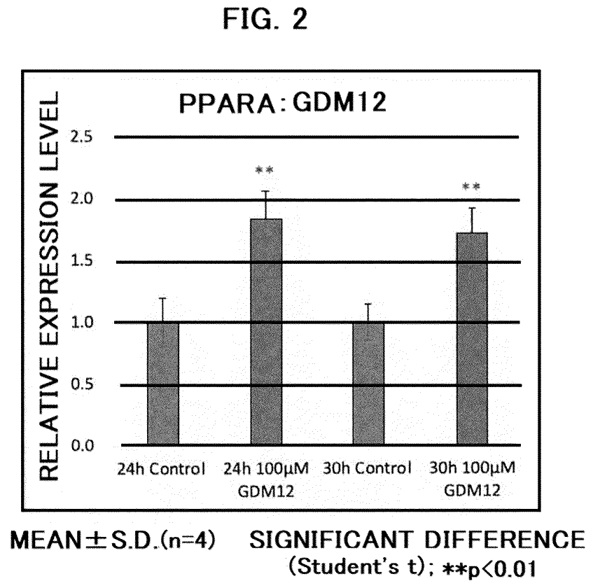

FIG. 1 and FIG. 2 are graphs illustrating the relative expression levels of PPARA genes in GDS23 and GDM12, respectively. The expression level in GDS23 was confirmed to be about 2.2 times that of the control, and the expression level in GDM12 was confirmed to be about 1.7 to 1.8 times that of the control.

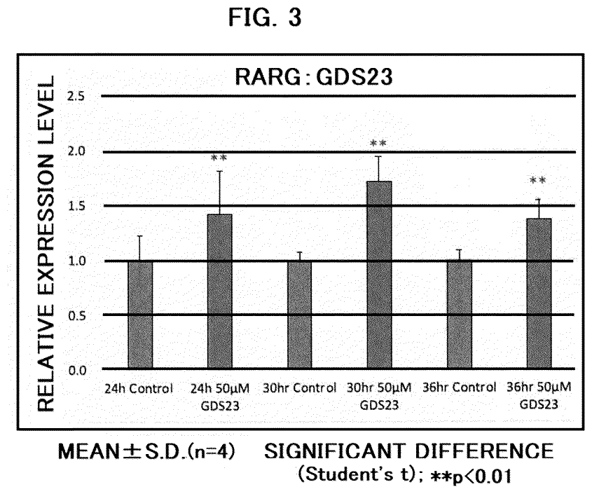

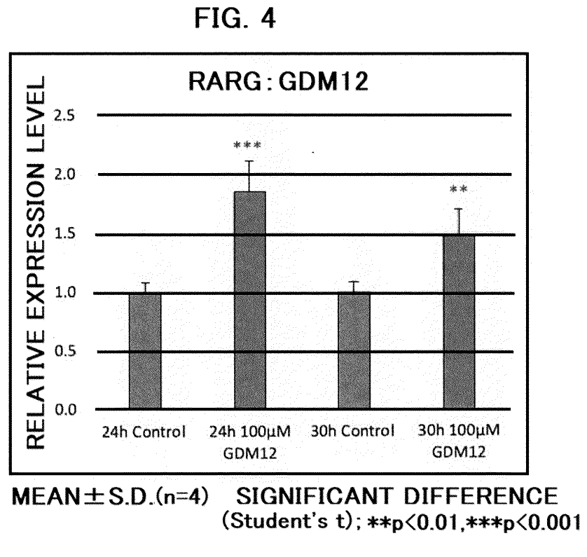

FIG. 3 and FIG. 4 are graphs illustrating the relative expression levels of the RARG genes in GDS23 and GDM12, respectively. The expression level in GDS23 was confirmed to be about 1.3 to 1.7 times that of the control, and the expression level in GDM12 was confirmed to be about 1.5 to 1.8 times that of the control.

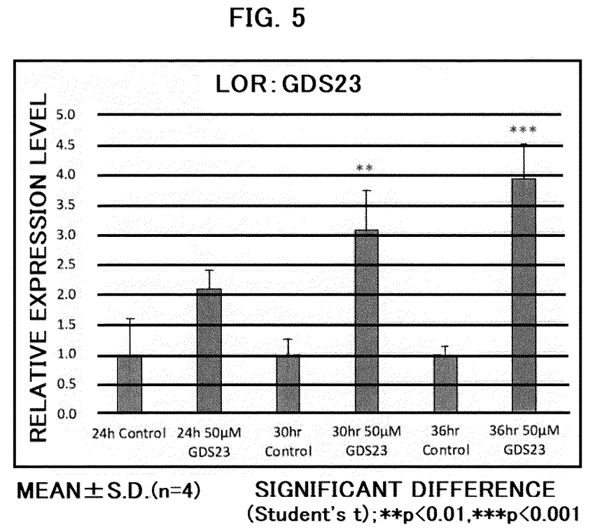

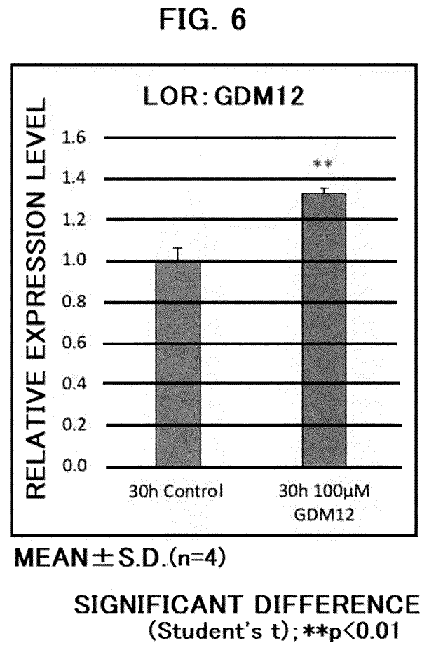

FIG. 5 and FIG. 6 are graphs illustrating the relative expression levels of LOR genes in GDS23 and GDM12, respectively. The expression level in GDS23 was confirmed to be about 2.0 to 3.9 times that of the control. The expression level in GDM12 was confirmed to be about 1.3 times that of the control.

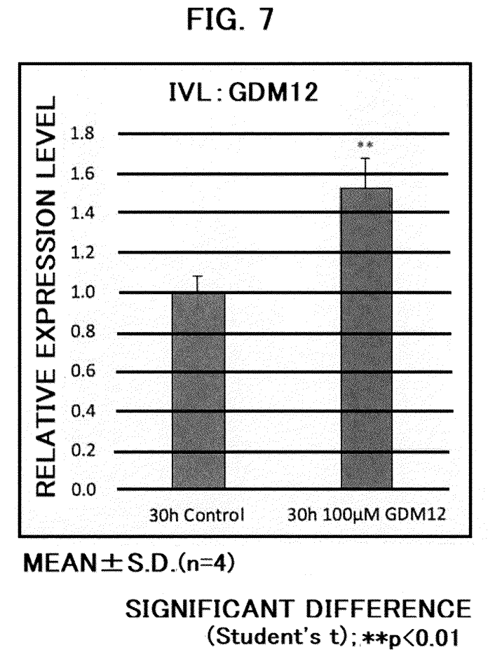

FIG. 7 is a graph illustrating the relative expression levels of IVL genes in GDM12. The expression level was confirmed to be about 1.5 times that of the control.

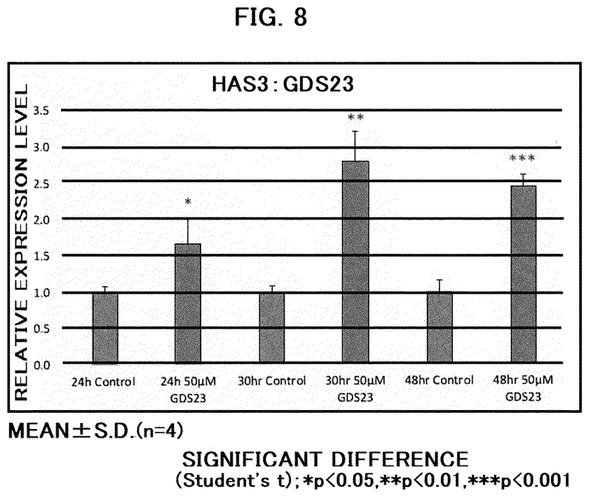

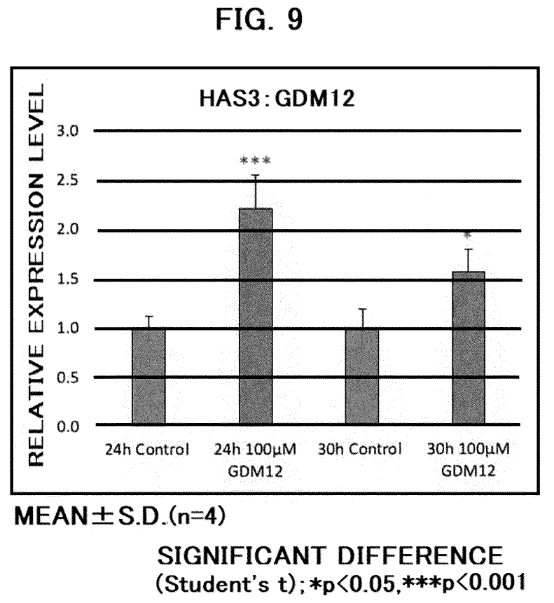

FIG. 8 and FIG. 9 are graphs illustrating the relative expression levels of HAS3 genes in GDS23 and GDM12, respectively. The expression level in GDS23 was confirmed to be about 1.7 to 2.8 times that of the control. The expression level in GDM12 was confirmed to be about 1.6 to 2.2 times that of the control.

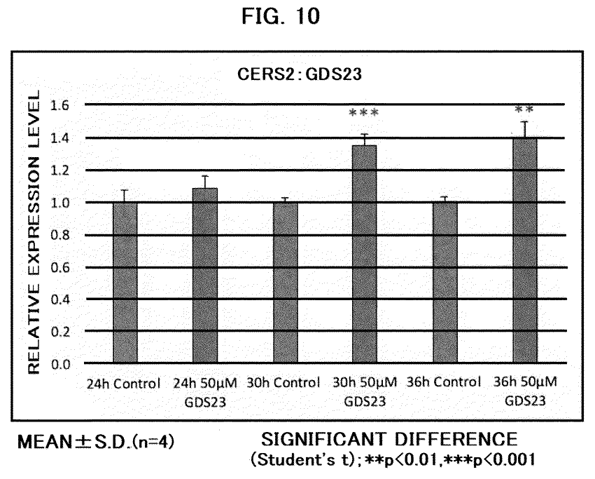

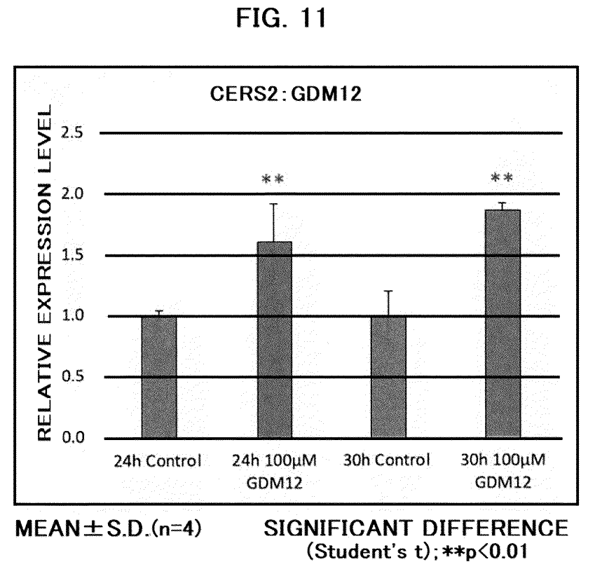

FIG. 10 and FIG. 11 are graphs illustrating the relative expression levels of CERS2 genes in GDS23 and GDM12, respectively. The expression level in GDS23 was confirmed to be about 1.1 to 1.4 times that of the control. The expression level in GDM12 was confirmed to be about 1.6 to 1.8 times that of the control.

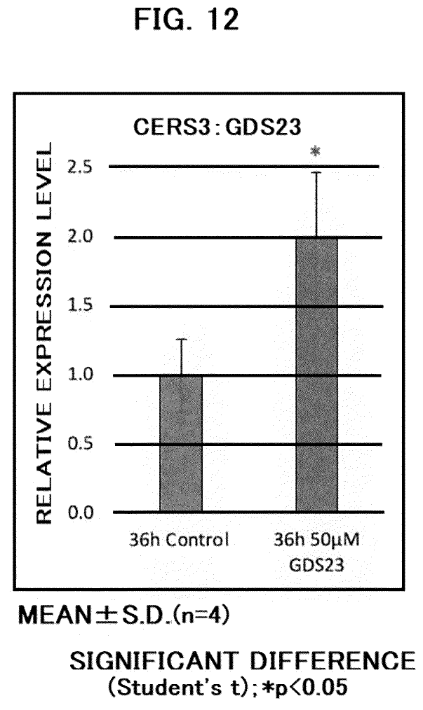

FIG. 12 is a graph illustrating the relative expression levels of CERS3 genes in GDS23. The expression level was confirmed to be about 2 times that of the control.

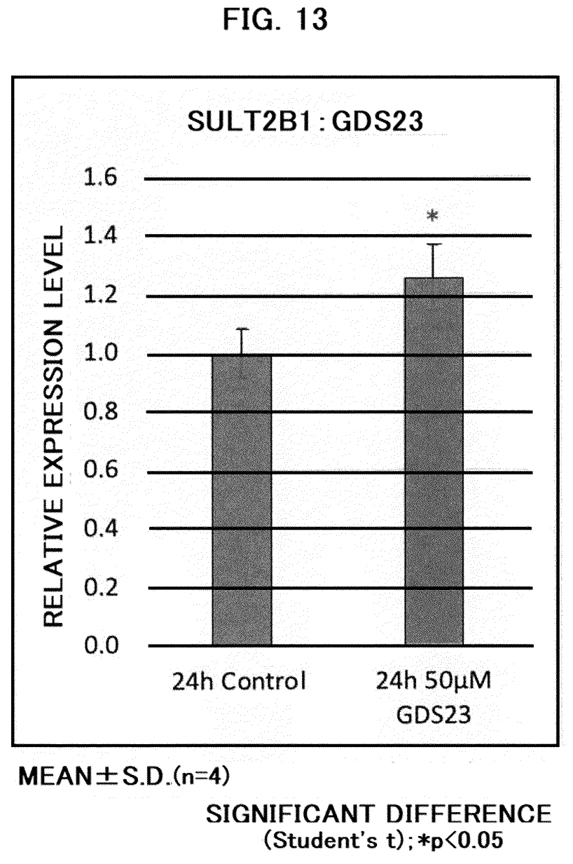

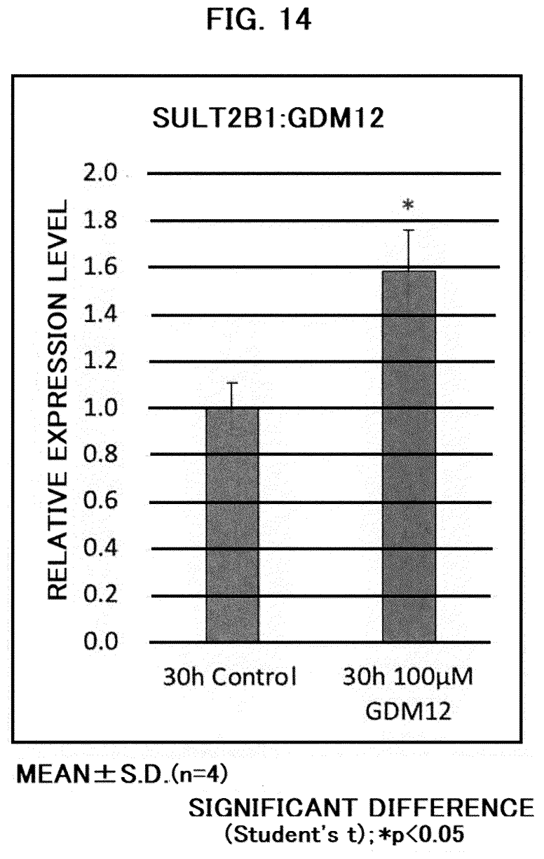

FIG. 13 and FIG. 14 are graphs illustrating the relative expression levels of SULT2B1 genes in GDS23 and GDM12, respectively. The expression level in GDS23 was confirmed to be about 1.2 times that of the control. The expression level in GDM12 was confirmed to be about 1.6 times that of the control.

### (2) Test for Detecting Production of Loricrin

A test for detecting the production of loricrin encoded by the LOR genes was performed using two staining methods: an immunostaining method using an anti-loricrin antibody and a hematoxylin-eosin staining method.

### (2-1) Test Method

Treatment of epidermal model Three-dimensional cultured human epidermal model (LabCyte EPI-MODEL24 6D: manufactured by Japan Tissue Engineering Co., Ltd.) was cultured in a medium (assay medium: manufactured by Japan Tissue Engineering Co., Ltd.) at 37° C. for 24 hours.

Thereafter, the medium was exchanged, and 50 μL of a sample prepared with purified water (2% preparation solution of GDS23) was applied to the surfaces of the stratum corneum, followed by continuous culture for 24 hours. The control for comparison was applied with purified water only.

Subsequently, the sample on the surface of the stratum corneum of the epidermal model was sucked up with a sterilized cotton swab to remove the excess sample, and then the epidermal model was continuously cultured without newly applying the sample on the surface of the stratum corneum, and the epidermal model was collected 9 days after the sample was applied.

### Preparation of Frozen Section of Epidermal Model and Loricrin Immunostaining Method

A frozen section was prepared from three-dimensional cultured skin tissues in which the collected epidermal model was embedded in a frozen tissue embedding agent (0. C. T. compound: manufactured by Sakura Finetek Japan Co., Ltd.), fixed in a 4% formaldehyde solution, and then blocked in a 3% BSA/PBS (-) solution containing 10% Normal Goat serum for 1 hour.

After the blocking agent was aspirated, an anti-loricrin antibody (manufactured by Proteintech Japan) (diluted 50 times with a 3% BSA/PBS solution) was added to the section, and the primary antibody reaction was carried out overnight at 4° C. Thereafter, a Goat Anti-Rabbit IgG H&L (Alexa Fluor (registered trademark) 488 antibody (manufactured by Abcam PLC) (diluted 250 times with a 3% BSA/PBS solution) was added thereto, and the secondary antibody reaction was carried out for 1 hour at room temperature in a dark place. And then, nuclear staining was performed with DAPI diluted 200 times with a 3% BSA/PBS solution. A fluorescence microscope (BZ-X810, KEYENCE) was then used to observe the green and blue fluorescence. The green fluorescence indicates the presence of loricrin. The blue fluorescence stains the nuclei of the epidermal model to confirm that the cells are normal.

### Preparation of Frozen Section of Epidermal Model and Hematoxylin-Eosin Staining Method

The recovered epidermal model was embedded in a frozen tissue embedding agent (0. C. T. compound: manufactured by Sakura Finetek Japan Co., Ltd.), to prepare a frozen section, which was fixed with a 4% formaldehyde solution and washed with purified water. The section was then immersed in a hematoxylin solution and stained. The section was washed with running water and then immersed in an eosin solution. Further, the section was dehydrated in 90% ethanol and 100% ethanol, and then cleared with xylene, and sealed. Thereafter, the section was then observed. The hematoxylin-eosin staining method was performed to confirm that there were no abnormalities in the cells of the epidermal model.

### (2-2) Test Results

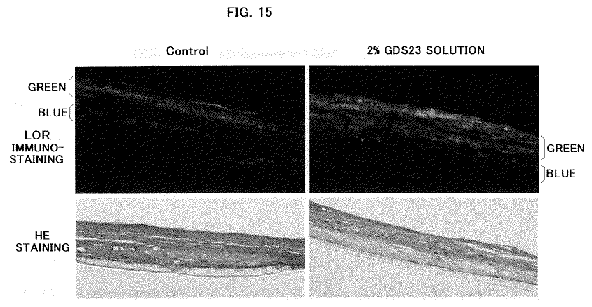

FIG. 15 illustrates the test results of the loricrin immunostaining method (upper row) and the hematoxylin-eosin staining method (lower row), respectively.

The green fluorescence in the upper fluorescence microscope image (the upper part of the band extending from the upper left to the lower right) indicates that the production amount of loricrin in the stratum corneum of the epidermal model coated with the GDS23 solution is higher than that of the control epidermal model.

The blue fluorescence (the lower part of the band extending from the upper left to the lower right) in the fluorescence microscope image in the upper row and the image of the hematoxylin-eosin staining method in the lower row indicate that there is no abnormality in the cells of the epidermal model.

### (3) Preparation of Cosmetic and Skin External Preparation

In the following descriptions, preparation examples of a cosmetic and a skin external preparation containing an expression amplifier for epidermal barrier function-related genes containing a diacylglycerol PEG adduct as an active ingredient are shown below. Each of the following preparation examples contains retinoic acid, retinol, a retinol derivative, and/or a retinoic acid derivative. Retinoic acid can bind to and activate the nuclear receptor RARγ. Each substance other than retinoic acid can act on RARγ by being converted into retinoic acid through hydrolysis, enzymatic action, or the like in the body.

As described above, the expression of RARγ is enhanced by the diacylglycerol PEG adduct in the present invention. Therefore, it is expected that the activation of RARγ is further promoted by the enrichment of retinoic acid in the body by the above-mentioned substance such as retinoic acid administered from the outside.

### (3-1) Preparation Example of Lotion

The lotion of preparation example 1 contains PEG-12 glycerol dimyristate, PEG-23 glycerol distearate, tocopheryl retinoate, retinol, retinol palmitate, and hydroxypinacolone retinoate.

TABLE 2
Preparation example 1 (lotion)

| Ingredients | Mass (%) |
|---|---|
| Water | 71.30% |
| Glycerin | 12.00% |
| BG | 8.00% |
| PEG-12 glycerol dimyristate | 3.00% |
| PEG-23 glycerol distearate | 3.00% |
| Squalane | 1.50% |
| Tocopheryl retinoate | 0.01% |
| Retinol | 0.03% |
| Retinol palmitate | 0.01% |
| Hydroxypinacolone retinoate | 0.01% |
| Caprylic/capric triglyceride | 0.25% |
| Dimethyl Isosorbide | 0.09% |
| Allantoin | 0.10% |
| Cholesterol | 0.10% |
| Tocopherol | 0.01% |
| Phenoxyethanol | 0.50% |
| EDTA-2Na | 0.09% |

### (3-2) Preparation Example of Milky Lotion

The milky lotion of preparation example 2 contains PEG-12 glycerol dimyristate, PEG-23 glycerol distearate, tocopheryl retinoate, retinol, and hydroxypinacolone retinoate.

TABLE 3
Preparation example 2 (milky lotion)

| Ingredients | Mass (%) |
|---|---|
| Water | 60.00% |
| PEG-12 glycerol dimyristate | 10.00% |
| Glycerin | 8.00% |
| BG | 6.00% |
| Caprylic/capric triglyceride | 4.40% |
| Squalane | 3.00% |
| Stearyl alcohol | 3.00% |
| Niacinamide | 1.00% |
| PEG-23 glycerol distearate | 0.50% |
| Tocopheryl retinoate | 0.30% |
| Retinol | 0.30% |
| Hydroxypinacolone retinoate | 0.10% |
| Cholesteryl stearate | 0.30% |
| Cholesterol | 0.10% |
| Tocopherol | 0.05% |
| Bitter orange flower oil | 0.02% |
| Arginine | 0.20% |
| Dimethyl Isosorbide | 0.90% |
| Ethylhexylglycerin | 0.30% |
| (Dimethicone/vinyl dimethicone) crosspolymer | 0.65% |
| Carbomer | 0.30% |
| Phenoxyethanol | 0.50% |
| EDTA-2Na | 0.08% |

### (3-3) Preparation Example of Aqueous Gel

The aqueous gel of preparation example 3 contains PEG-23 glycerol distearate, PEG-12 glycerol dimyristate, tocopheryl retinoate, retinol, and hydroxypinacolone retinoate.

TABLE 4
Preparation example 3 (aqueous gel)

| Ingredients | Mass (%) |
|---|---|
| Water | 64.60% |
| PEG-23 glycerol distearate | 10.00% |
| Glycerin | 10.00% |
| Caprylic/capric triglyceride | 4.40% |
| Stearyl alcohol | 4.00% |
| Cyclohexylglycerin | 1.00% |
| Pentyleneglycol | 1.00% |
| Niacinamide | 1.00% |
| PEG-12 glycerol dimyristate | 0.50% |
| Tocopheryl retinoate | 0.30% |
| Retinol | 0.30% |
| Hydroxypinacolone retinoate | 0.10% |
| (PEG-240/decyl tetradeceth-20/HDI) copolymer | 1.00% |
| Allantoin | 0.10% |
| Panthenol | 0.10% |
| Dimethyl Isosorbide | 0.90% |
| Citric acid | 0.02% |
| Sodium citrate | 0.08% |
| Lavender oil | 0.10% |
| Phenoxyethanol | 0.50% |

### (3-4) Preparation Example of Cream

The cream of preparation example 4 contains PEG-23 glycerol distearate, PEG-12 glycerol dimyristate, tocopheryl retinoate, hydroxypinacolone retinoate, and retinol.

TABLE 5
Preparation example 4 (cream)

| Ingredients | Mass (%) |
|---|---|
| Water | 50.00% |
| Propanediol | 12.00% |
| Squalane | 10.00% |
| PEG-23 glycerol distearate | 5.00% |
| Caprylic/capric triglyceride | 4.40% |
| Stearyl alcohol | 3.00% |
| Cetearyl alcohol | 3.00% |
| Dimethyl isosorbide | 2.70% |
| Dimethicone | 2.00% |
| Behenyl alcohol | 2.00% |
| Glyceryl stearate | 1.60% |
| PEG-12 glycerol dimyristate | 1.00% |
| Tocopheryl retinoate | 0.30% |
| Hydroxypinacolone retinoate | 0.30% |
| Retinol | 0.30% |
| Tocopherol acetate | 0.10% |
| Sodium methyl stearoyl taurate | 0.80% |
| Cholesteryl stearate | 0.40% |
| Polyglyceryl stearate-10 | 0.80% |
| Citric acid | 0.02% |
| Sodium citrate | 0.08% |
| Phenoxyethanol | 0.10% |
| EDTA-2Na | 0.10% |

### (3-5) Preparation Example of Ointment

The ointment of preparation example 5 contains PEG-23 glycerol distearate, PEG-12 glycerol dimyristate, tocopheryl retinoate, hydroxypinacolone retinoate, and retinoic acid.

TABLE 6
Preparation example 5 (ointment)

| Ingredients | Mass (%) |
|---|---|
| Water | 28.00% |
| Glycerin | 18.00% |
| Propanediol | 15.00% |
| PEG-23 glycerol distearate | 12.50% |
| Cetearyl alcohol | 9.00% |
| Dimethicone | 5.00% |
| Caprylic/capric triglyceride | 3.60% |
| Trimethylolpropane triethylhexanoate | 3.00% |
| Dimethyl isosorbide | 2.70% |
| PEG-12 glycerol dimyristate | 1.00% |
| Tocopheryl retinoate | 0.30% |
| Hydroxypinacolone retinoate | 0.30% |
| Retinoic acid | 0.10% |
| Dimethicone crosspolymer | 0.45% |
| (Dimethicone/vinyl dimethicone) crosspolymer | 0.40% |
| Citric acid | 0.02% |
| Sodium citrate | 0.08% |
| Phenoxyethanol | 0.50% |
| EDTA-2Na | 0.05% |

### (3-6) Preparation Example of Retinoic Acid-Containing Lotion

The retinoic acid-containing lotion of preparation example 6 contains PEG-12 glycerol dimyristate, PEG-23 glycerol distearate, tocopheryl retinoate, and retinoic acid.

TABLE 7
Preparation example 6 (retinoic acid-containing lotion)

| Ingredients | Mass (%) |
|---|---|
| Water | 75.50% |
| Glycerin | 10.00% |
| BG | 6.00% |
| PEG-12 glycerol dimyristate | 3.00% |
| PEG-23 glycerol distearate | 3.00% |
| Squalane | 1.50% |
| Tocopheryl retinoate | 0.01% |
| Retinoic acid | 0.01% |
| Caprylic/capric triglyceride | 0.18% |
| Allantoin | 0.10% |
| Cholesterol | 0.10% |
| Tocopherol | 0.01% |
| Phenoxyethanol | 0.50% |
| EDTA-2Na | 0.09% |

### (3-7) Preparation Example of Retinoic Acid-Containing Cream

The retinoic acid-containing cream of preparation example 7 contains PEG-23 glycerol distearate, PEG-12 glycerol dimyristate, retinoic acid, tocopheryl retinoate, and retinol.

TABLE 8
Preparation example 7 (retinoic acid-containing cream)

| Ingredients | Mass (%) |
|---|---|
| Water | 55.00% |
| Propanediol | 10.00% |
| Squalane | 10.00% |
| PEG-23 glycerol distearate | 5.00% |
| Caprylic/capric triglyceride | 4.40% |
| Stearyl alcohol | 3.00% |
| Cetearyl alcohol | 3.00% |
| Dimethicone | 2.00% |
| Behenyl alcohol | 2.00% |
| Glyceryl stearate | 1.60% |
| PEG-12 glycerol dimyristate | 1.00% |
| Retinoic acid | 0.10% |
| Tocopheryl retinoate | 0.30% |
| Retinol | 0.20% |
| Tocopherol acetate | 0.10% |
| Sodium methyl stearoyl taurate | 0.80% |
| Cholesteryl stearate | 0.40% |
| Polyglyceryl stearate-10 | 0.80% |
| Citric acid | 0.02% |
| Sodium citrate | 0.08% |
| Phenoxyethanol | 0.10% |
| EDTA-2Na | 0.10% |

Although not exemplified, in the cosmetic or the skin external preparation, there are various combinations of the diacylglycerol PEG adduct and other ingredients in addition to those described above. In addition, in a case where retinoic acid, retinol, a retinol derivative, or a retinoic acid derivative is contained as an ingredient, only one or a combination of a plurality of these ingredients is selected as necessary.

Although the present invention has been described with reference to the embodiments, the present invention is not limited to these embodiments, and modifications obvious from these embodiments are also included in the present invention.

1. A method for amplifying expression of an epidermal barrier function-related gene, the method comprising applying a diacylglycerol PEG adduct having a following structural formula; 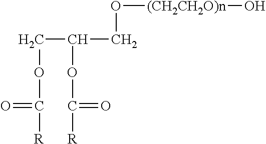
in which number of carbon atoms of R in a long-chain fatty acid is in a range of 11 to 23 and n in a polyethylene glycol chain is in a range of 11 to 46,
wherein the diacylglycerol PEG adduct is at least one of a group consisting of PEG-12 glycerol dimyristate (GDM12), PEG-12 glycerol distearate (GDS12), PEG-23 glycerol distearate (GDS23), PEG-23 glycerol dipalmitate (GDP23), and PEG-12 glycerol dioleate (GDO12).

2. A method for using an active ingredient for amplifying expression of an epidermal barrier function-related gene in a cosmetic or a skin external preparation, the method comprising using, as the active ingredient, a diacylglycerol PEG adduct having a following structural formula; 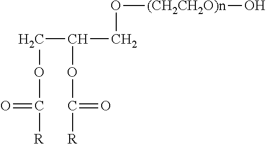
in which number of carbon atoms of R in a long-chain fatty acid is in a range of 11 to 23 and n in a polyethylene glycol chain is in a range of 11 to 46,
wherein the diacylglycerol PEG adduct is at least one of a group consisting of PEG-12 glycerol dimyristate (GDM12), PEG-12 glycerol distearate (GDS12), PEG-23 glycerol distearate (GDS23), PEG-23 glycerol dipalmitate (GDP23), and PEG-12 glycerol dioleate (GDO12).

3. The method according to claim 1, wherein the epidermal barrier function-related gene is at least one of a group consisting of PPARA, RARG, LOR, IVL, HAS3, CER2, CER3, and SULT2B1.

4. The method according to claim 1, wherein the diacylglycerol PEG adduct permeates into epidermis in a solution state or a vesicle state.

5. The method according to claim 2, wherein the cosmetic or the skin external preparation further comprises at least one of a group consisting of retinoic acid, retinol, a retinol derivative, and a retinoic acid derivative.
